(19) United States
(12) Patent Application Publication
(10) Pub. No.: US 2026/0213538 A1
(43) Pub. Date: Jul. 23, 2026

(54) SYSTEM AND METHOD FOR BALANCING AN ELECTRICAL POWER SYSTEM USING METHANOL

(71) Applicant: TOPSOE A/S, Kgs. Lyngby (DK)

(72) Inventors: Niels Ulrik ANDERSEN, Gentofte (DK); Jes Nikolaj KNUDSEN, Birkerød (DK)

(73) Assignee: TOPSOE A/S, Kgs. Lyngby (DK)

(21) Appl. No.: 19/139,658

(22) PCT Filed: Dec. 20, 2023
(86) PCT No.: PCT/EP2023/086878

§ 371 Date: Jun. 16, 2025

(30) Foreign Application Priority Data
Dec. 22, 2022 (DK) PA202201195

(51) Int. Cl. H02J 3/28; C07C 29/151; C25B 1/04; C25B 15/08; H02J 3/38; H02J 15/50; H02J 101/22; H02J 101/28; H02J 101/30; H02J 101/35

(52) U.S. Cl. CPC H02J 3/28; C07C 29/151; C25B 1/04; C25B 15/081; H02J 3/381; H02J 15/50; H02J 2101/22; H02J 2101/28; H02J 2101/30; H02J 2101/35

(57) ABSTRACT

A power balancing system and process is provided, in which a first electrolysis unit (10) outputs a first hydrogen rich stream (11), which is converted in a methanol synthesis plant (20) to a first methanol-rich stream (21). A methanol storage unit (40) receives and stores the first methanol-rich stream (21). When additional electrical power is required, methanol from the methanol storage unit (40) can be used for power generation. The system and process allow excess electrical power to be converted into and stored as methanol during periods of low demand, and used to generate electrical power when demand is higher.

### TECHNICAL FIELD

A power balancing system and process is provided, in which a first electrolysis unit outputs a first hydrogen rich stream, which is converted in a methanol synthesis plant to a first methanol-rich stream. A methanol storage unit receives and stores the first methanol-rich stream. When additional electrical power is required, methanol from the methanol storage unit can be used in power generation in various ways. The system and process allow excess electrical power to be converted into and stored as methanol during periods of low demand, and used to generate electrical power when demand is higher.

### BACKGROUND

Levelling out power production and consumption and/or price is important when all or part of power is generated from a renewable source (wind, solar, etc). In the increasing rollout of renewable power generation, account needs to be taken for the variability in power production from such sources, which can occur hourly, daily or seasonally. Large-scale battery arrays are being commercially developed as one solution to this problem, in an attempt to balance or “level out” the supply and demand curves.

A system for balancing the power generation from renewable energy and the grid consumption using ammonia is described in U.S. Pat. No. 10,323,544.

At the same time as balancing the output from renewable power, there is a focus on reducing output of CO₂ and other waste gases in downstream processes. Elimination of combustion and cracking steps could also lead to reduced heat requirement, and/or the need to re-use heated streams elsewhere in a plant. There is also a need to increase power generation, while reducing plot area and utilities'consumption. It would also be advantageous if power balancing could be combined with useful electrochemical synthesis processes (“power-to-X”).

These goals are addressed by the present technology.

### SUMMARY

The present idea is to produce methanol (MeOH) from renewable power (wind, solar, etc.) when power is in excess. MeOH is produced from H₂ produced by electrolysis from renewable power and CO₂ from storage or another source. The MeOH can easily be stored and converted to power when renewable power source is insufficient to supply the power grid.

So, in a first embodiment the present invention relates to a power balancing system, said system comprising:
a water-rich feed,
optionally, an external CO₂ feed,
a supply of renewable electricity;
a first electrolysis unit
a methanol synthesis plant
a methanol storage unit, and
a CO₂ storage section
wherein,
the first electrolysis unit is arranged to receive the water-rich feed and at least a first portion of said supply of renewable electricity, and to output a first hydrogen rich stream;
the methanol synthesis plant is arranged to receive at least a portion of the first hydrogen rich stream, and a CO₂ stream from the CO₂ storage section and/or the external CO₂ feed, and to output a first methanol-rich stream;
the methanol storage unit is arranged to receive and store at least a portion of the first methanol-rich stream; and output a second methanol-rich stream; and wherein the system is arranged to generate electrical power from said second methanol-rich stream, via one or more of the following:
   wherein the system comprises a methanol fuel cell, the methanol fuel cell being arranged to receive at least a first portion of the second methanol-rich stream from the methanol storage unit and to output a first electrical power stream;
   wherein the system comprises a methanol cracker, a first CO₂ removal section, and a first power generation section, the methanol cracker being arranged to receive at least a second portion of the second methanol-rich stream from the methanol storage unit and to output a combined H₂ and CO₂ stream; the first CO₂ removal section being arranged to receive at least a portion of the combined H₂ and CO₂ stream and to output a first CO₂-rich stream and a second hydrogen rich stream; the first power generation section being arranged to receive at least a portion of the second hydrogen rich stream from the CO₂ removal section and combust it so as to output a second electrical power stream;
   wherein the system comprises a methanol cracker, a first CO₂ removal section, and a hydrogen fuel cell, the methanol cracker being arranged to receive at least a second portion of the second methanol-rich stream from the methanol storage unit and to output a combined H₂ and CO₂ stream; the first CO₂ removal section being arranged to receive at least a portion of the combined H₂ and CO₂ stream and to output a first CO₂-rich stream and a second hydrogen rich stream; the hydrogen fuel cell being arranged to receive at least a portion of the second hydrogen rich stream from the CO₂ removal section and to output a third electrical power stream;
   wherein the system further comprises a second power generation section, and a second CO₂ removal section, the second power generation section being arranged to receive at least a third portion of the second methanol-rich stream from the methanol storage unit and combust it so as to output a fourth electrical power stream and a flue gas stream, the second CO₂ removal section being arranged to receive the flue gas stream from the second power generation section and output a second CO₂-rich stream and a balance stream containing nitrogen, oxygen and water;
   or wherein system further comprises a methanol dehydration section, and a diesel generator, said methanol dehydration section being arranged to receive at least a fourth portion of the second methanol-rich stream from the methanol storage unit and dehydrate it to provide a dimethyl ether (DME) stream; and wherein said diesel generator is arranged to receive at least a portion of said dimethyl ether stream and combust it so as to output a fifth electrical power stream;
and wherein at least one of the first, second, third, fourth or fifth electrical power streams are arranged to be fed to a power grid.

A process for balancing electrical power in a power balancing system as described herein is also provided. Further details of the technology are provided in the following claims, description text and the appended figures.

### LEGENDS

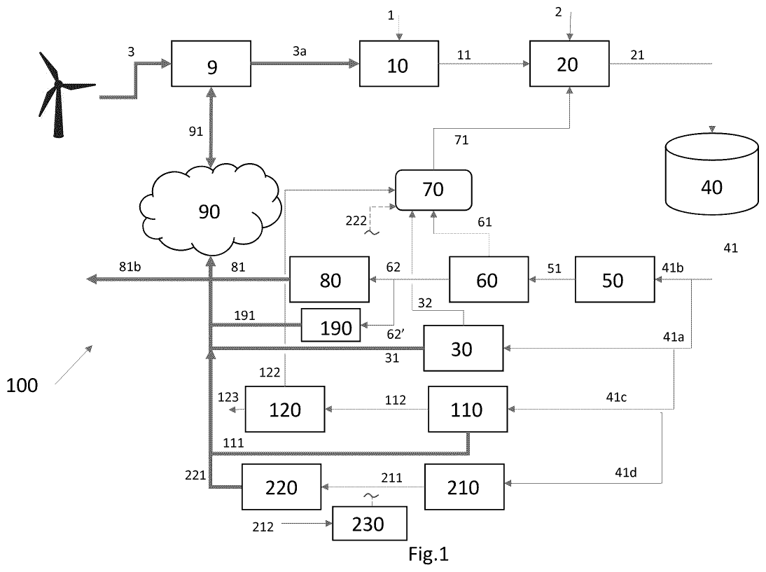

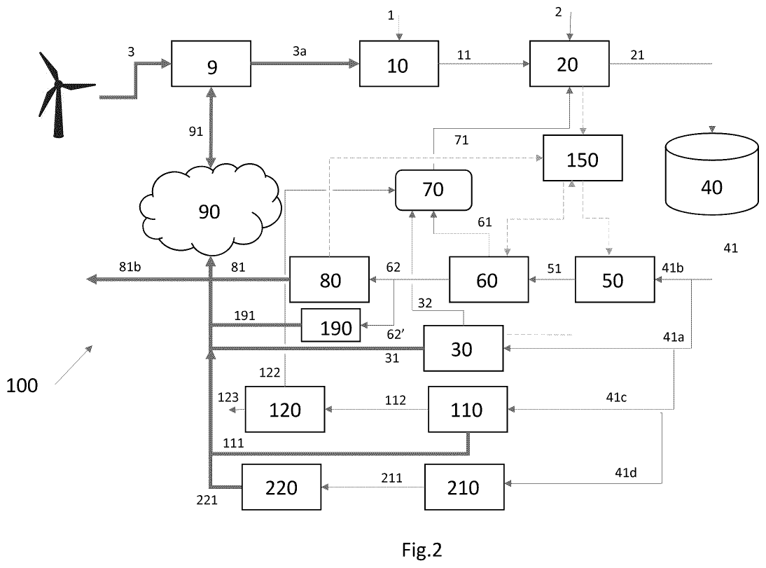

FIG. 1 shows a schematic layout of a power balancing system according to one embodiment of the invention FIG. 2 shows a schematic layout of a power balancing system according to a further embodiment of the invention

### DETAILED DISCLOSURE

A power balancing system is provided. The system allows excess electrical power to be converted into and stored as methanol during periods of low demand, and used to generate electrical power when demand is higher.

When a stream is described as “rich” in a certain component, it is generally meant that the majority (i.e. over 50% by volume) of the stream consists of said component. Depending on the stream in question, the proportion of the component may be higher.

In general terms, the system comprises:
a water-rich feed,
optionally, an external CO₂ feed,
a supply of renewable electricity;
a first electrolysis unit
a methanol synthesis plant
a methanol storage unit, and
a CO₂ storage section

A supply of renewable electricity is required. Renewable electricity may come from solar, wind, wave or tidal energy, and is typically intermittent. A portion of the supply of renewable electricity is provided to the first electrolysis unit.

The water-rich feed is suitably high purity (e.g. over 98%, or over 99%) water or steam. The first electrolysis unit (which is preferably one or more SOECs) is arranged to receive the water-rich feed and at least a first portion of said supply of renewable electricity, and to output a first hydrogen rich stream, by electrolysis of the first water-rich feed.

The first hydrogen rich stream produced by the first electrolysis unit is suitably high purity (e.g. over 98%, or over 99%) hydrogen.

The methanol synthesis plant (also called a “methanol loop”) is arranged to receive at least a portion of the first hydrogen rich stream, and a CO₂ stream from the CO₂ storage section and/or the external CO₂ feed, and to output a first methanol-rich stream. The skilled person knows how to design and implement a methanol synthesis plant to produce methanol from H2 and CO₂.

The first methanol-rich stream is suitably high-purity (e.g. over 98%, or over 99%) methanol. To obtain such high purity, the methanol synthesis plant may comprise a purification section, in which the purity of raw methanol is increased.

The extent to which an external CO2 feed is required depends-inter alia-on the amount of CO₂ stored in the CO₂ storage section, and the amount of methanol conversion required. This depends-in turn-on the amount of CO₂ produced elsewhere in the system. If the amount of CO2 stored in the CO2 storage section is insufficient, external CO₂ feed may be used. The external CO₂ feed may be a biogas feed, or a CO₂ from a flue gas (possibly generated by one or more other components of the system).

The methanol storage unit is arranged to receive and store at least a portion of the first methanol-rich stream; and to output a second methanol-rich stream. First and second methanol-rich streams are essentially identical in composition, but may vary in terms of their physical properties, e.g. temperature or pressure. Methanol storage typically takes place in large, pressurised tanks.

As noted, the methanol from the methanol storage unit can be used in a number of ways, dictated by demand from the power grid. The system is arranged to generate electrical power from the second methanol-rich stream, suitably via one or more of the following aspects.

In a first aspect, the system comprises a methanol fuel cell. The methanol fuel cell is arranged to receive at least a first portion of the second methanol-rich stream from the methanol storage unit and output a first electrical power stream. Fuel cells which convert methanol to electrical power are known. A by-product of the methanol fuel cell is a water-rich stream, at least a portion of which may be provided as feed to the first electrolysis unit. The methanol fuel cell typically also outputs a third CO₂-rich stream.

In a second aspect, the system comprises a methanol cracker, a first CO₂ removal section, a and a first power generation section. The methanol cracker is arranged to receive at least a second portion of the second methanol-rich stream from the methanol storage and water unit and to output a combined stream of approx. 1 mole % methanol, 20 mole % CO2 65mole % H2, 10 mole % H2O. Cracking of methanol is typically a high-temperature catalysed process. The combined H₂ and CO₂ stream is passed to the first CO₂ removal section, where CO₂ is separated, e.g. in a cryogenic process. CO₂ can also be removed from the combined cracker effluent gas by conventional CO₂ removal system (amine wash, membrane, etc) at elevated pressure (“pre-combustion”).

Removal of CO₂ at this stage avoids a build-up of excess CO₂ in a downstream combustion process. The first CO₂ removal section outputs a first CO₂-rich stream (typically comprising more than 90 vol %, suitably more than 95 vol % CO₂) and a second hydrogen rich stream (typically comprising more than 90 vol %, suitably more than 95 vol % H₂).

The first power generation section is arranged to receive at least a portion of the second hydrogen rich stream from the CO₂ removal section and combust it so as to output a second electrical power stream. Electrical power may be generated for instance by a steam Rankine cycle of another Rankine cycle. The first power generation section suitably comprises a combustion section arranged to combust the second hydrogen rich stream and a turbine, arranged to convert heat from the combustion section into electrical power.

In a third aspect, the system comprises a methanol cracker, a first CO₂ removal section (in the same manner as the second aspect), and a hydrogen fuel cell. The methanol cracker is arranged to receive at least a second portion of the second methanol-rich stream from the methanol storage unit and to output a combined H₂ and CO₂ stream, in the same way as for the second aspect, above. The first CO₂ removal section is arranged to receive at least a portion of the combined H₂ and CO₂ stream and to output a first CO₂-rich stream and a second hydrogen rich stream (in a similar manner to the second aspect, above) Instead of the second hydrogen-rich stream being combusted, as in the second aspect, the hydrogen fuel cell is arranged to receive at least a portion of the second hydrogen rich stream from the CO₂ removal section and to output a third electrical power stream.

In a fourth aspect, the system further comprises a second power generation section, and a second CO₂ removal section. In this third aspect, the methanol is combusted directly, in the second power generation section, to provide electrical power. The second power generation section suitably comprises a combustion section arranged to combust the second methanol-rich stream and a turbine, arranged to convert heat from the combustion section into electrical power.

In other words, the second power generation section is arranged to receive at least a third portion of the second methanol-rich stream from the methanol storage unit and combust it so as to output a fourth electrical power stream and a flue gas stream. The second CO₂ removal section is arranged to receive the flue gas stream from the second power generation section and output a second CO₂-rich stream and a balance stream. If pure O₂ is used for combustion, then the balance stream is water-rich, e.g. over 90% water, or even over 95% water. If air is used for the combustion, which is more likely, then the balance stream will additionally comprise nitrogen, argon and oxides of nitrogen.

In a fifth aspect, system further comprises a methanol dehydration section, and a diesel generator. The methanol dehydration section is arranged to receive at least a fourth portion of the second methanol-rich stream from the methanol storage unit and dehydrate it to provide a dimethyl ether (DME) stream. DME can be combusted directly in a diesel generator. Therefore, the diesel generator is arranged to receive at least a portion of said dimethyl ether (DME) stream and combust it so as to output a fifth electrical power stream.

A third CO₂ removal section may be arranged to receive a flue gas stream from the diesel generator and output a fourth CO₂-rich stream, and optionally to feed said fourth CO₂-rich stream to said CO₂ storage section.

All aspects, set out above, may be implemented separately, as alternatives. A system may also comprise two or more aspects, e.g. three aspects, four aspects or all five aspects together.

In all aspects, at least one of the first, second, third, fourth or fifth electrical power streams are arranged to be fed to a power grid. Feeding electrical power back to the grid allows power to be balanced via methanol production, storage and conversion back into electricity.

In one embodiment, the first, second, third, fourth or fifth electrical power streams are arranged to increase in response to a drop in the supply of renewable electricity. This can be achieved by increasing the supply of the second methanol-rich stream from the methanol storage unit, in each aspect. In other words, an increase in the first, second, third, fourth or fifth electrical power streams may be provided by increased output of the second methanol-rich stream from said methanol storage unit.

In one aspect, the methanol fuel cell is also arranged to output a water-rich stream, and to feed at least a portion of the water-rich stream as feed to the first electrolysis unit.

The CO₂ storage section may receive CO₂ from one or more other sections or units in the system. For instance, the CO₂ storage section may further be arranged to receive at least a portion of the first CO₂-rich stream, at least a portion of the second CO₂-rich stream, and/or at least a portion of the third CO₂-rich stream. The CO₂ may be stored as liquid or as solid CO₂.

In one particular aspect, illustrated in FIG. 2, the system further comprises a heat integration system. The heat integration system receives heat energy from components of the system which generate heat, and supplies heat energy to components of the system which require heat. Therefore, the heat integration system may be arranged to receive heat energy from one or more of: the methanol synthesis plant, the first power generation section, the methanol fuel cell, and the second power generation section, and to provide heat energy to one or more of: the methanol cracker, the first CO₂ removal section and/or the second CO₂ removal section.

The system may further comprise a power regulating section, arranged between the supply of renewable energy and the first electrolysis unit. The power regulating section is arranged to send any excess electrical power from the supply of renewable energy and the first electrolysis unit, e.g. when supply outstrips grid demand.

A process for balancing electrical power in a power balancing system is also provided. In general terms, the process comprises the steps of:
providing the system as set out herein,
feeding the water-rich feed and at least a first portion of said supply of renewable electricity to the first electrolysis unit and outputting a first hydrogen rich stream;
feeding at least a portion of the first hydrogen rich stream, and a CO₂ stream from the CO₂ storage section and/or the external CO₂ feed to the methanol synthesis plant and outputting a first methanol-rich stream;
feeding at least a portion of the first methanol-rich stream; the methanol storage unit and outputting—on demand—a second methanol-rich stream;
and wherein, the process further comprises one or more steps of:
   feeding at least a first portion of the second methanol-rich stream from the methanol storage unit to the methanol fuel cell, and outputting a first electrical power stream;
   feeding at least a second portion of the second methanol-rich stream from the methanol storage unit to the methanol cracker, and outputting a combined H₂ and CO₂ stream; and feeding at least a portion of the combined H₂ and CO₂ stream to a first CO₂ removal section, and outputting a first CO₂-rich stream and a second hydrogen rich stream; and feeding at least a portion of the second hydrogen rich stream from the CO₂ removal section to the first power generation section and combusting it so as to output a second electrical power stream;
   feeding at least a second portion of the second methanol-rich stream from the methanol storage unit to the methanol cracker, and outputting a combined H₂ and CO₂ stream; and feeding at least a portion of the combined H₂ and CO₂ stream to a first CO₂ removal section, and outputting a first CO₂-rich stream and a second hydrogen rich stream; and feeding at least a portion of the second hydrogen rich stream from the CO₂ removal section to the hydrogen fuel cell and outputting a third electrical power stream;
   feeding at least a third portion of the second methanol-rich stream from the methanol storage unit to the second power generation section and combusting it so as to output a fourth electrical power stream and a flue gas stream, and feeding the flue gas stream from the second power generation section to the second CO₂ removal section and outputting a second CO₂-rich stream and balance steam containing mostly Nitrogen, Water and Oxygen;
   feeding at least a fourth portion of the second methanol-rich stream from the methanol storage unit to the methanol dehydration section and dehydrating it to provide a dimethyl ether stream; and feeding at least a portion of the DME stream to the diesel generator and combusting it so as to output a fifth electrical power stream;
followed by a step of feeding at least one of the first, second, third, fourth or fifth electrical power streams to a power grid.

All details of the system of the invention are relevant to the process of the invention, mutatis mutandis. For instance, the process suitably comprises a step of increasing the first, second, third, fourth or fifth electrical power streams in response to a drop in the supply of renewable electricity. As above, an increase in the first, second, third, fourth or fifth electrical power streams may be provided by increased output of the second methanol-rich stream from said methanol storage unit.

In one aspect of the process, the methanol fuel cell is also arranged to output a third CO₂-rich stream, said process comprising the further steps of feeding at least a portion of the first CO₂-rich stream, at least a portion of the second CO₂-rich stream, and/or at least a portion of the third CO₂-rich stream to the CO₂ storage section.

The methanol fuel cell may also be arranged to output a water-rich stream, said process comprising the further steps of feeding at least a portion of the water-rich stream as feed to the first electrolysis unit.

### Specific Embodiments

FIG. 1 illustrates a power balancing system 100. The first electrolysis unit 10 receives the water-rich feed 1 and at least a first portion 3 of the supply of renewable electricity 3, and outputs a first hydrogen rich stream 11. The methanol synthesis plant 20 receives at least a portion of the first hydrogen rich stream 11, and a CO₂ stream 71 from the CO₂ storage section 70 and/or the external CO₂ feed 2, and outputs a first methanol-rich stream 21.

The methanol storage unit 40 receives and stores at least a portion of the first methanol-rich stream 21; and outputs a second methanol-rich stream 41. The system 100 is arranged to generate electrical power from the second methanol-rich stream 41, via one or more of the following routes:
When the system 100 comprises a methanol fuel cell 30, the methanol fuel cell 30 receives at least a first portion 41a of the second methanol-rich stream 41 from the methanol storage unit 40 and outputs a first electrical power stream 31.

When the system 100 comprises a methanol cracker 50, a first CO₂ removal section 60, and a first power generation section 80, the methanol cracker 50 receives at least a second portion 41b of the second methanol-rich stream 41 from the methanol storage unit 40 and outputs a combined H₂ and CO₂ stream 51. The first CO₂ removal section 60 receives at least a portion of the combined H₂ and CO₂ stream 51 and outputs a first CO₂-rich stream 61 and a second hydrogen rich stream 62. The first power generation section 80 receives at least a portion of the second hydrogen rich stream 62 from the CO₂ removal section 60 and combusts it so as to output a second electrical power stream 81.

When the system 100 comprises a methanol cracker 50, a first CO₂ removal section 60, and a hydrogen fuel cell 190, the methanol cracker 50 receives at least a second portion 41b of the second methanol-rich stream 41 from the methanol storage unit 40 and outputs a combined H₂ and CO₂ stream 51. The first CO₂ removal section 60 receives at least a portion of the combined H₂ and CO₂ stream 51 and outputs a first CO₂-rich stream 61 and a second hydrogen rich stream 62. The hydrogen fuel cell 190 receives at least a portion 62′ of the second hydrogen rich stream 62 from the CO₂ removal section 60 and outputs a third electrical power stream 191.

When the system 100) further comprises a second power generation section 110, and a second CO₂ removal section 120, the second power generation section 110 receives at least a third portion 41c of the second methanol-rich stream 41 from the methanol storage unit 40 and combusts it so as to output a fourth electrical power stream 111 and a flue gas stream 112. The second CO₂ removal section 120 receives the flue gas stream 112 from the second power generation section 110 and outputs a second CO₂-rich stream 122 and a balance stream 123.

When the system 100 further comprises a methanol dehydration section 210, and a diesel generator 220, the methanol dehydration section 210 receives at least a fourth portion 41d of the second methanol-rich stream 41 from the methanol storage unit 40 and dehydrates it to provide a dimethyl ether (DME) stream 211. The diesel generator 220 receives at least a portion of said dimethyl ether (DME) stream 211 and combusts it so as to output a fifth electrical power stream 221.

At least one of the first, second, third, fourth or fifth electrical power streams 31, 81, 111, 191, 221 are arranged to be fed to a power grid 90.

While the invention has been described with reference to a number of embodiments and aspects, the overall scope of the invention is defined in the appended claims. The skilled person may combine embodiments and aspects as required, within the scope of the invention. All documents mentioned herein are incorporated by reference.

1. A power balancing system said system comprising:
a water-rich feed,
optionally, an external CO₂ feed,
a supply of renewable electricity;
a first electrolysis unit
a methanol synthesis plant
a methanol storage unit, and
a CO₂ storage section
wherein,
the first electrolysis unit is arranged to receive the water-rich feed and at least a first portion of said supply of renewable electricity, and to output a first hydrogen rich stream;
the methanol synthesis plant is arranged to receive at least a portion of the first hydrogen rich stream and a CO₂ stream from the CO₂ storage section and/or the external CO₂ feed, and to output a first methanol-rich stream
the methanol storage unit is arranged to receive and store at least a portion of the first methanol-rich stream; and output a second methanol-rich stream; and wherein,
the system is arranged to generate electrical power from said second methanol-rich stream, via one or more of the following:
wherein the system comprises a methanol fuel cell the methanol fuel cell being arranged to receive at least a first portion of the second methanol-rich stream from the methanol storage unit and to output a first electrical power stream;
wherein the system comprises a methanol cracker, a first CO₂ removal section, and a first power generation section, the methanol cracker being arranged to receive at least a second portion of the second methanol-rich stream from the methanol storage unit and to output a combined H₂ and CO₂ stream; the first CO₂ removal section being arranged to receive at least a portion of the combined H₂ and CO₂ stream and to output a first CO₂-rich stream and a second hydrogen rich stream; the first power generation section being arranged to receive at least a portion of the second hydrogen rich stream from the CO₂ removal section and combust it so as to output a second electrical power stream;
wherein the system comprises a methanol cracker, a first CO₂ removal section, and a hydrogen fuel cell, the methanol cracker being arranged to receive at least a second portion of the second methanol-rich stream from the methanol storage unit and to output a combined H₂ and CO₂ stream; the first CO₂ removal section being arranged to receive at least a portion of the combined H₂ and CO₂ stream and to output a first CO₂-rich stream and a second hydrogen rich stream; the hydrogen fuel cell being arranged to receive at least a portion of the second hydrogen rich stream from the CO₂ removal section and to output a third electrical power stream;
wherein the system further comprises a second power generation section, and a second CO₂ removal section, the second power generation section being arranged to receive at least a third portion of the second methanol-rich stream from the methanol storage unit and combust it so as to output a fourth electrical power stream and a flue gas stream, the second CO₂ removal section being arranged to receive the flue gas stream from the second power generation section and output a second CO2-rich stream and a balance stream
or wherein system further comprises a methanol dehydration section, and a diesel generator, said methanol dehydration section being arranged to receive at least a fourth portion of the second methanol-rich stream from the methanol storage unit and dehydrate it to provide a dimethyl ether stream; and wherein said diesel generator is arranged to receive at least a portion of said dimethyl ether stream and combust it so as to output a fifth electrical power stream;
and wherein at least one of the first, second, third, fourth or fifth electrical power streams are arranged to be fed to a power grid.

2. The system according to claim 1, wherein the first, second, third, fourth or fifth electrical power streams are arranged to increase in response to a drop in the supply of renewable electricity.

3. The system according to claim 2, wherein an increase in the first, second, third, fourth or fifth electrical power streams is provided by increased output of the second methanol-rich stream from said methanol storage unit.

4. The system according to claim 1, wherein the first power generation section comprises a combustion section arranged to combust the second hydrogen rich stream and a turbine, arranged to convert heat from the combustion section into electrical power.

5. The system according to claim 1, wherein the methanol fuel cell also outputs a water-rich stream.

6. The system according to claim 1, wherein the methanol dehydration section being arranged to output a water stream, and wherein at least a portion of said water stream is arranged to be fed to the first electrolysis unit.

7. The system according to claim 1, wherein the methanol fuel cell also outputs a third CO2-rich stream.

8. The system according to claim 1, wherein the CO₂ storage section is further arranged to receive at least a portion of the first CO₂-rich stream, at least a portion of the second CO₂-rich stream, and/or at least a portion of the third CO₂-rich stream.

9. The system according to claim 1, further comprising a heat integration system said heat integration system arranged to receive heat energy from the methanol synthesis plant, the first power generation section, the methanol fuel cell, and/or the second power generation section, and to provide heat energy to the methanol cracker, the first CO₂ removal section and/or the second CO₂ removal section.

10. The system according to claim 1, wherein the external CO₂ feed is a biogas feed, or a CO₂ from a flue gas.

11. The system according to claim 1, comprising a power regulating section, arranged between the supply of renewable energy and the first electrolysis unit.

12. The system according to claim 1, further comprising a third CO₂ removal section being arranged to receive a flue gas stream from the diesel generator and output a fourth CO₂-rich stream, and optionally to feed said fourth CO₂-rich stream to said CO₂ storage section.

13. A process for balancing electrical power in the power balancing system according to claim 1, said process comprising the steps of:
providing the system,
feeding the water-rich feed and at least a first portion of said supply of renewable electricity to the first electrolysis unit and outputting a first hydrogen rich stream;
feeding at least a portion of the first hydrogen rich stream, and a CO₂ stream from the CO₂ storage section and/or the external CO₂ feed to the methanol synthesis plant and outputting a first methanol-rich stream;
feeding at least a portion of the first methanol-rich stream; the methanol storage unit and outputting—on demand—a second methanol-rich stream;
and wherein, the process further comprises one or more steps of:
feeding at least a first portion of the second methanol-rich stream from the methanol storage unit to the methanol fuel cell, and outputting a first electrical power stream;
feeding at least a second portion of the second methanol-rich stream from the methanol storage unit to the methanol cracker, and outputting a combined H₂ and CO₂ stream; and feeding at least a portion of the combined H₂ and CO₂ stream to a first CO₂ removal section, and outputting a first CO₂-rich stream and a second hydrogen rich stream; and feeding at least a portion of the second hydrogen rich stream from the CO₂ removal section to the first power generation section and combusting it so as to output a second electrical power stream;
feeding at least a second portion of the second methanol-rich stream from the methanol storage unit to the methanol cracker, and outputting a combined H₂ and CO₂ stream; and feeding at least a portion of the combined H₂ and CO₂ stream to a first CO₂ removal section, and outputting a first CO₂-rich stream and a second hydrogen rich stream; and feeding at least a portion of the second hydrogen rich stream from the CO₂ removal section to the hydrogen fuel cell and outputting a third electrical power stream;
feeding at least a third portion of the second methanol-rich stream from the methanol storage unit to the second power generation section and combusting it so as to output a fourth electrical power stream and a flue gas stream, and feeding the flue gas stream from the second power generation section to the second CO2 removal section and outputting a second CO₂-rich stream and a balance stream;
feeding at least a fourth portion of the second methanol-rich stream from the methanol storage unit to the methanol dehydration section and dehydrating it to provide a dimethyl ether stream; and feeding at least a portion of the DME stream to the diesel generator and combusting it so as to output a fifth electrical power stream;
followed by a step of feeding at least one of the first, second, third, fourth or fifth electrical power streams to a power grid.

14. The process according to claim 13, said process comprising a step of increasing the first, second, third, fourth or fifth electrical power streams in response to a drop in the supply of renewable electricity.

15. The process according to claim 14, wherein an increase in the first, second, third, fourth or fifth electrical power streams is provided by increased output of the second methanol-rich stream from said methanol storage unit.

16. The process according to claim 13, wherein the methanol fuel cell is also arranged to output a third CO₂-rich stream, said process comprising the further steps of feeding at least a portion of the first CO₂-rich stream at least a portion of the second CO₂-rich stream, and/or at least a portion of the third CO₂-rich stream to the CO₂ storage section.

17. The process according to claim 13, wherein the methanol fuel cell is also arranged to output a water-rich stream, said process comprising the further steps of feeding at least a portion of the water-rich stream as feed to the first electrolysis unit.
